(19) United States
(12) Patent Application Publication
(10) Pub. No.: US 2003/0165586 A1
(43) Pub. Date: Sep. 4, 2003

(54) Anti-angiogenic composition comprising ticlopidine and gingko biloba extract

(76) Inventors: Min-Young Kim, Daejeon (KR); Byung-Young Park, Daejeon (KR); Chang-Hee Moon, Daejeon (KR)

Correspondence Address: David A Einhorn, Anderson Kill & Olick, 1251 Avenue of the Americas, New York, NY, 10020-1182, US

(21) Appl. No.: 10/343,146

(22) PCT Filed: Jul. 27, 2001
(86) PCT No.: PCT/KR01/01280

(51) Int. Cl. A61K 35/78; A61K 31/4743

(52) U.S. Cl. 424/752; 514/301

(57) ABSTRACT

A composition comprising ticlopidine and a Ginkgo biloba extract is used in the manufacture of a medicine for inhibiting angiogenesis, which has an enhanced anti-angiogenic activity with reduced side effect.

### FIELD OF THE INVENTION

The present invention relates to a use of a composition comprising ticlopidine and a Ginkgo biloba extract in the manufacture of a medicine having enhanced anti-angiogenic activity with reduced cytotoxicity.

### BACKGROUND OF THE INVENTION

Angiogenesis is the process of generating new capillary blood vessels. Neovascularization is tightly regulated, and the proliferation rate of endothelial cells is very low compared with that of other cell types in the body. The failure to regulate angiogenesis may lead to such diseases as rheumatoid arthritis, diabetic retinopathy, psoriasis and tumor (Liekens S., et al., Biochem. Pharmacol., 61, 253-270 (2001); and Folkman J., Nat. Med., 1, 27-31 (1995)). Solid tumor growth and metastasis, in particular, are angiogenesis-dependent. That is, new blood vessels in tumor provide not only nutrients and oxygen but also a way for tumor cells to enter the blood stream causing metastasis.

Currently, a large variety of chemotherapeutic drugs are used for the treatment of cancer. However, many compounds show severe side effects and limited efficacy, due to the lack of tumor selectivity and the development of drug resistance. Since anti-angiogenic therapy targets activated endothelial cells, it offers several advantages in terms of selectivity and efficacy.

The entrapment of tumor cells in the blood clots during disseminated intravascular coagulation or in microthrombi may lead to tumor cell lodgment in the microcirculation. Therefore, the use of anitithrombotic drugs is a viable strategy for cancer metastasis therapy.

Ticlopidine, 5-[(2-chlorophenyl)methyl]-4,5,6,7-tetrahydrothieno[3,2-C]pyridine, is a platelet aggregation inhibitor with a broad scope of clinical application (Ferrara N., et al., Nat. Med., 5, 1359-1364 (1999)). Ticlopidine lowers the fibrinogen level in plasma and exerts the effect of improving the plasticity of red blood cells (Ferrara N., et al., vide supra; and Klein-Soyer C., et al., J. Cell. Physiol., 160, 316-322 (1994)). A recent report has shown that thienopyridine derivatives have anti-angiogenic effect on endothelial cells (Gryglewski R. J., et al., Eur. J. Pharmacol., 308, 61-67 (1996)). However, the anti-angiogenic efficacy of ticlopidine is very low, requiring a high dose thereof for obtaining any significant anti-angiogenic effect. According to another report, ticlopidine does not significantly influence the metastasis although it inhibits platelet aggregation (Fabra A., et al., Invasion metastasis, 7, 53-60 (1987)). That is, ticlopidine is weakly anti-angiogenic, and it is not usable by itself as a primary anti-angiogenic drug because of its undesirable side effect.

Therefore, the present inventor has endeavored to develop an improved anti-angiogenic drug and unexpectedly found that the combined use of a Ginkgo biloba extract with ticlopidine brings about the synergistic effects of enhancing the anti-angiogenic activity and reducing the cellular toxicity of ticlopidine.

### SUMMARY OF THE INVENTION

Accordingly, it is a primary object of the present invention to provide a novel use of a composition comprising ticlopidine and a Ginkgo biloba extract for inhibiting angiogenesis which has enhanced anti-angiogenic activity and causes no adverse side effect.

In accordance with one aspect of the present invention, there is provided a use of a composition comprising ticlopidine and a Ginkgo biloba extract in the manufacture of a medicine for inhibiting angiogenesis.

### BRIEF DESCRIPTION OF THE DRAWINGS

The above objects and features of the present invention will become apparent from the following description of preferred embodiments taken in conjunction with the accompanying drawings, in which:

FIGS. 1A to ID show the results of tube formation of HUVEC in Matrigel untreated and treated with 50 μM ticlopidine, 10 μg/ml Ginkgo biloba extract, and 25 μM ticlopidine together with 5 μg/ml Ginkgo biloba extract, respectively.

### DETAILED DESCRIPTION OF THE INVENTION

Ticlopidine may be chemically synthesized according to the process described by Albert Rene Joseph Gastaigne (U.S. Pat. No. 4,051,141) or commercially obtained. Ticlopidine may be used in an amount of 30 to 90% by weight, preferably 40 to 80% by weight, based on the weight of the composition.

Further, a Ginkgo biloba extract is prepared in accordance with any of the conventional extraction methods. For instance, 10 to 20 l of an aqueous alcohol, e.g., ethanol or methanol, or acetone is added to 1 kg of dried Ginkgo biloba leaves and the mixture is allowed to stand at a temperature ranging from 60 to 80° C., for a period ranging from 30 min. to 2 hours. This extraction process may be repeated 1 to 3 times. The resulting extract is concentrated to obtain a concentrated Ginkgo biloba extract. The Ginkgo biloba extract may be used in an amount of 10 to 70% by weight, preferably 20 to 50% by weight, based on the weight of the composition.

The combined use of ticlopidine and the Ginkgo biloba extract generates a remarkable synergistic effect of enhancing the anti-angiogenic activity beyond the level expected when ticlopidine or Ginkgo biloba extract is used alone. Therefore, a combination of ticlopidine and a Ginkgo biloba extract may be advantageously used in treating a disease caused by abnormal angiogenesis, e.g., angioma, angiofibroma, arthritis, diabetic retinopathy, premature infant's retinopathy, neovascular glaucoma, corneal disease, involutional macula, degeneration of macula, pterygium, retinal degeneration, retrolental fibroplasias, granular conjunctivitis, psoriasis, telangiectasis, pyogenic granuloma, seborrheic dermatitis, acne, cancer and metastasis.

Moreover, a Ginkgo biloba extract reduces the known ticlopidine-induced cellular toxicity, and the combined use of ticlopidine and a Ginkgo biloba extract does not lead to cell toxicity.

The present invention also provides a pharmaceutical composition for inhibiting angiogenesis, which comprises ticlopidine and a Ginkgo biloba extract as active ingredients, together with pharmaceutically acceptable excipients, carrier or diluents.

A pharmaceutical formulation may be prepared in accordance with any of the conventional procedures. In preparing the formulation, the active ingredients are preferably admixed or diluted with a carrier, or enclosed within a carrier, which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of ticlopidine may range from about 0.5 to 15.0 mg/kg body weight, preferably 2.0 to 10.0 mg/kg body weight, and that of a Ginkgo biloba extract may range from about 0.1 to 20.0 mg/kg body weight, preferably 0.5 to 4.0 mg/kg body weight, which can be administered in a single dose or in divided doses.

However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The following Examples are intended to further illustrate the present invention without limiting its scope.

### EXAMPLE 1

### Tube Formation Assay

Human umbilical vein endothelial cells (HUVECs) were isolated from freshly obtained cords digested with 0.1% collagenase (Sigma) (Grant D. S., et al., Cell, 58, 933-943 (1989)). The cells were grown in M199 medium containing 20% fetal bovine serum, ECGS, heparin and penicillin-streptomycin. Cells between 3 to 5 passages were used in the following experiments.

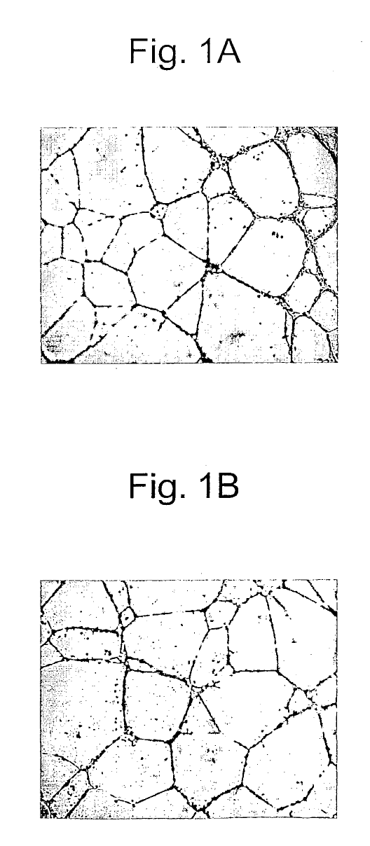

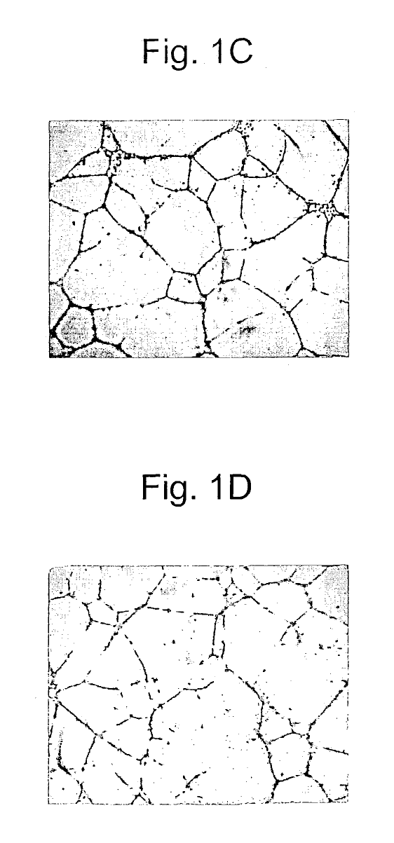

The tube formation assay was performed as follows: 48-well plates were coated with 0.2 ml of Matrigel (BD Bioscience, Bedford, Mass., USA) and incubated at 37° C. for 1 hr. 4˜6×10⁴ HUVECs resuspended in 0.4 ml of M199 medium were added to each well. Ticlopidine (Sigma Chemical Co., USA) and Ginkgo biloba extract (Hwa Il Pharmaceutical Co., LTD, Korea), a standardized extract that contained 24% flavonoid glycosides and 6% terpenoids, were added to the Matrigel. Final concentration of ticlopidine and Ginkgo biloba extract were 25 μM and 5 μg/ml, respectively. As a control, the procedure was repeated without added ticlopidine and Ginkgo biloba extract. Comparative runs were also conducted by the same procedure using ticlopidine alone (comparative run 1) or only with Ginkgo biloba extract (comparative run 2). The extent of tube formation was determined by measuring the total tube area per well relative to that of the control using image analysis program Image-Pro Pluso (Media Cybernetics, USA). Results are shown in FIGS. 1A to 1D and Table I.

TABLE I

| | Total Tube Area % |
|---|---|
| Control | 100 |
| 50 μM Ticlopidine (Comparative run 1) | 91 |
| 10 μg/ml Ginkgo biloba extract (Comparative run 2) | 89 |
| 25 μM Ticlopidine + 5 μg/ml Ginkgo biloba extract | 66 |

As can be seen from FIGS. 1A to 1D and Table I, ticlopidine together with Ginkgo biloba extract showed an inhibitory effect on the HUVEC tube formation which is much higher than that expected based on the comparative runs. Thus, the combined use of a Ginkgo biloba extract brings about a remarkable synergistic effect of enhancing the anti-angiogenic effect of the ticlopidine.

### EXAMPLE 2

### Cell Cytotoxicity Assay

The viability of HUVECs and normal keratinocyte cell line, HaCaT cells (College of Medicine, Seoul National University) was tested with cell proliferation kit II (Roche, Germany). The results showed that the number of viable cells was reduced by treatment with ticlopidine as a dose-dependent manner. That is, ticlopidine was cytotoxic and LC₅₀ was 250 μM and 750 μM for HaCaT cells and HUVECs, respectively. In combined treatment of ticlopidine with Ginkgo biloba extract, 25 μM of ticlipidine was sufficient for the inhibition of angiogenesis, which did not significantly affect the cell viability.

### EXAMPLE 3

### Matrigel Plug Assay

The anti-angiogenic activity of a mixture of ticlopidine and Ginkgo biloba extract was confirmed in the Matrigel plug assay.

0.4 ml portion of Matrigel each mixed with 50 ng/ml of basic fibroblast growth factor (bFGF) and 50 units of heparin were implanted by subcutaneous injection in 5-week-old C57BL/6 female mice, and a mixture of 0.65 mg ticlopidine and 0.4 mg Ginkgo biloba extract was orally administrated to each mouse twice per day for four days. Then each mouse was sacrificed and the Matrigel was excised. For comparison, the procedure was repeated using ticlopidine or Ginkgo biloba extract alone. The formation of blood vessels in Matrigel was tested by measuring the amount of hemoglobin (Hb) in the Matrigel using the Drabkin reagent kit (Sigma Chemical Co., St. Louise, Mich., USA) which contained standards of known amounts of Hb.

The result in Table II unequivocally shows that the formation of new blood vessel in Matrigel was drastically reduced by the synergism generated by the combined use of ticlopidine and the Ginkgo biloba extract.

TABLE II

| | Dose (mg/mouse) | Hemoglobin (%) |
|---|---|---|
| Control | (−) | 100 |
| Ticlopidine | 0.65 | 95 |
| Ginkgo biloba extract | 0.4 | 32 |
| Ticlopidine + | 0.65 + 0.4 | 9 |
| Ginkgo biloba extract | | |

While the subject invention has been described and illustrated with reference to the preferred embodiments only, it may be apparent to those skilled in the art that various changes and modifications can be made therein without departing from the spirit and scope of the present invention which is defined in the appended claims.

What is claimed is

1. A use of a composition comprising ticlopidine and a Ginkgo biloba extract in the manufacture of a medicine for inhibiting angiogenesis.

2. The use of claim 1, wherein the composition comprises 30 to 90% by weight of ticlopidine and 10 to 70% by weight of the Ginkgo biloba extract, based on the weight of the composition.

3. The use of claim 1, which is used for treating a disease selected from the group consisting of angioma, angiofibroma, arthritis, diabetic retinopathy, premature infant's retinopatlhy, neovascular glaucoma, corneal disease, involutional macula, degeneration of macula, pterygium, retinal degeneration, retrolental fibroplasias, granular conjunctivitis, psoriasis, telangiectasis, pyogenic granuloma, seborrheic dermatitis, acne, cancer and metastasis.
